(19) United States
(12) Patent Application Publication
(10) Pub. No.: US 2003/0226695 A1
(43) Pub. Date: Dec. 11, 2003

(54) Weight control method using physical activity based parameters

(76) Inventor: James R. Mault, Evergreen, CO (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/296,656

(22) PCT Filed: May 24, 2001
(86) PCT No.: PCT/US01/16877

(51) Int. Cl. G01G 19/40; A61B 10/00; G06F 17/00

(52) U.S. Cl. 177/25.16; 128/921

(57) ABSTRACT

A method of assisting a person to achieve a weight control goal comprises determining a resting energy expenditure for the person using an indirect calorimeter, and converting the resting energy expenditure of the person into a number of resting points. The activity level of the person can be monitored or estimated, and converted into a number of activity points. The sum of activity points and resting points can be compared with the number of diet points consumed by the person, wherein the diet points are calculated based on calorie content and other nutritional values. The balance of these weight control points can be presented to a person, so as to assist the person succeed in a weight control program.

### FIELD OF THE INVENTION

The invention relates to the definition and use of physical activity based parameters, for example in weight loss and physical fitness programs.

### BACKGROUND OF THE INVENTION

Weight control is of great importance to a large number of people. However, calorie counting can be difficult for the average person. The numbers involved are often in the hundreds or thousands, and become difficult to record, add, and otherwise become familiar with. Equations and tables exist for converting exercises into calories expended, but these can also be tiresome to use, and errors can easily occur. Also, these equations and tables are only correct for a person with typical physiology, for a person of average demographics and physiology. Gender corrections are available, but these often alone are not sufficient to provide accurate data.

Recently, Miller-Kovach et al. (U.S. Pat. No. 6,040,531) described the definition and use of diet based parameters, which they called diet points. These diet points are used in weight control programs, such as those supervised by Weight Watchers Inc., and are calculated from the calorie, fat, and fiber content of food consumed. Further details are found in U.S. Pat. No. 6,040,531, incorporated herein by reference.

An equation for diet points, from U.S. Pat. No. 6,040,531, is given below:

$${P}_{D} = \frac{c}{{k}_{1}} + \frac{f}{{k}_{2}} - \frac{r}{{k}_{3}} \qquad ( Equation 1 )$$

where c is calorie content in kcal, f is the fat content in grams, and r is the fiber content in grams. The fiber term (in r) can be omitted.

The coefficients k₁, k₂, and k₃ are chosen so that a typical person on a weight control program handles small integers, for example in the range 0 to 100, when dealing with daily calorie consumption. The coefficients are further chosen to encourage healthy eating habits, in that a person is encouraged to eat high fiber, low fat foods. Typical values of the coefficients, as disclosed by Miller-Kovach et al., are k₁=20, k₂=12, and k₃=5, giving the following equation:

$${P}_{D} = \frac{c}{20} + \frac{f}{12} - \frac{r}{5} \qquad ( Equation 2 )$$

In a weight control program according to the disclosure of Miller-Kovach et al., a person is assigned a daily diet points target based on their body weight. However, there are problems in using body weight to determine calorie needs. A person's calorie requirements can also change over time, due to processes not well correlated with body weight changes, particularly for a person on a limited calorie diet.

A person's calorie requirements are determined by their total energy expenditure (TEE), which is the sum of activity energy expenditure (AEE) and resting energy expenditure (REE), also known as resting metabolism. REE is correlated with lean body weight, and is not well correlated with total body weight as body fat percentage can vary significantly. A person enrolling in a weight control program may have a body fat percentage considerably above or below average, in which case their calorie or diet point allowance will be inaccurate.

Further, there are serious problems associated with attempting to control body weight by restricting calorie intake. A person's metabolic rate can slow, in response to a perceived threat of starvation. Muscle mass can be lost if the person's physical activity levels fall, and this will cause a further fall over a long time period of the person's metabolic rate. In this case, if the person's metabolic rate falls to a greater degree than their reduced calorie intake, weight can be gained even on a reduced calorie diet. This outcome would typically be viewed as unsatisfactory.

### SUMMARY OF THE INVENTION

An improved weight control method is described, in which the dietary intake, metabolic rate, and physical activity of a person are recorded using comparable weight control parameters, such as diet points, activity points, and resting energy expenditure points (or resting points). We will use the term diet point to refer to some number derived from nutrition information, such as calorie content, nutritional content, glycemic index, and the like. In a simple case, the diet parameter may be the calorie content of food. The diet point can be derived from a combination of nutritional data (as described in U.S. Pat. No. 6,040,531), for example using Equation 1 above.

Resting energy expenditure is more closely related to lean body weight than to total body weight. Fat cells add to body weight, but not significantly to resting metabolism. The improved weight control method preferably includes the measurement of metabolic rate using an indirect calorimeter. A suitable device for measuring metabolic rate is the GEM (Gas Exchange Monitor), invented by James R. Mault, which in a preferred embodiment comprises an oxygen fluorescence sensor and an ultrasonic bi-directional flow meter. The GEM measures the oxygen consumption of a person, allowing calculation of their metabolic rate. It is advantageous in a weight control program to use a measured metabolic rate of a person, rather than one estimated from body weight. Hence, an indirect calorimeter such as the GEM forms part of an improved weight loss program. Alternatively, body fat content can be determined (e.g. using calipers, buoyancy, or electrical conductance measurements) allowing resting energy expenditure to be better estimated using the lean body weight.

The improved weight control method allows simple translation of metabolic rate and physical activity levels to parameters comparable to those such as diet points used in measuring food consumption. The person using the improved method can maintain a current account balance in weight control parameters, such as diet points, by recording diet points related to the person's consumption of consumables (which includes eating, drinking, taking medicine, intravenous feeding, and other consumption methods), and comparable weight control parameters correlated with energy expenditure (expenditure parameters, or expenditure points). In a preferred embodiment, these expenditure points comprise resting points correlated with resting energy expenditure, and activity points correlated with physical activity levels of the person. A computing device can be used to monitor food intake and activity levels. A balance (or difference) between recorded diet parameters (such as diet points), and the sum of expenditure points (resting points and activity points) can be calculated at any time relative, and the balance compared with a target balance determined so as to allow the successful completion of a weight control program.

For example, a person may record diet points consumed (P_{D}) on a portable computing device. The resting energy expenditure of the person is determined using an indirect calorimeter, and converted to resting points, P_{R}. This value can be increased by a factor related to the lifestyle of the person. The value of P_{R} can also be determined from the measured energy expenditure of the person engaged in typical or sedentary activities. Further, activities (such as exercise programs) can be recorded by the person, and converted to activity points P_{A}. The difference between P_{D} and (P_{R}+P_{A}) can then be determined, and presented to the person on a display of the portable computing device. A clock on the portable computing device can be used to scale P_{R} to a fraction of a day (or other time period) for which the balance is computed. Hence, in an improved weight control program, the person carries a computing device to monitor or record the consumption of consumables and activity, and to view a current balance of diet points consumed and expenditure points earned. This computing device may be a PDA (personal digital assistant), other portable computing device, calculator, wireless telephone, wristwatch, entertainment device, modified glasses, helmet-mounted display, pager, physiological monitor, or other computing device. A desktop computer, or remote computer accessed over a network, can also be used for calculating calorie and/or point balances.

Activity levels of a person can be estimated using body mounted accelerometers. For example, a person might engage in an activity e.g. running on the spot, while wearing an accelerometer. The person's metabolic rate is determined using an indirect calorimeter or other metabolic rate meter. The signal from the accelerometer can then be correlated with a quantitative increase in metabolism, and hence to calorie burning rate. The time-dependent decrease of the metabolic rate after the activity has ceased can also be determined and included in a quantitative model of how activity relates to calorie expenditure, and the calorie expenditure converted to activity points.

The nutritional data of food consumed can be recorded using a software application program on a computing device, for example using a menu-based entry system, bar-code reader for reading package markings, direct data entry, wireless transmission from dispensing machines, etc. A suitable software program has been described by Williams in U.S. Pat. Nos. 5,704,350 and 4,891,756, incorporated herein by reference. A daily recommended diet point range can be defined as described in U.S. Pat. No. 6,040,531. However, in an improved method, a computing device is used to maintain a rolling balance of the weight control parameters. A total recommended dietary intake, in calories, diet points, or other diet parameters, is determined over a set period of time at the beginning of the weight loss program. This total intake is determined based on the person's resting energy expenditure, expected activity level, and intended weight change (if any).

On the first day of the weight control program, the computing device recommends meals based on the calculated average daily food intake over the duration of the program. In the improved weight control method, the computing device records the actual food consumed on the first and subsequent days of the program, allowing the recommended daily intake to be modified. Activity levels can also be recorded, and weight control goals can be recalculated if activity levels differ from predicted estimates. For example, if activity levels exceed the level predicted at the beginning of the program, then the average daily diet point allowance will be recalculated upwards, or a lower final weight goal may be suggested. If activity levels do not meet the level predicted, then the average allowed diet point intake can be reduced accordingly, and the person can be provided with encouraging feedback.

The person uses the computing device to call up a current account balance of weight control parameters. In one embodiment, weight control parameters are earned by activity and metabolic rate, and spent by eating food. If dietary intake has been restricted to the recommended level, but activity has exceeded the expected level, the balance will be positive. If dietary intake has exceeded the recommended level without a corresponding increase in activity, the balance will be negative. (This sign convention can be reversed if the person prefers). Eating contributes to a negative balance; exercise contributes to a positive balance. A negative balance should be paid off over the remainder of the program, by reducing food intake, or by extra exercise, or some combination. The current account balance model allows special occasions to be included in the weight loss plan. For example, the food allowance for a birthday can be set higher at the beginning of the program, and the allowance for other days made slightly lower. A person can increase the limit for one day, e.g. for a celebration, with compensation automatically calculated for the remainder of the program. Rigid daily limit weight loss programs do not allow such flexibility.

The weight control parameters can also be referred to as “diet dollars”. In an improved weight loss program, a person can be given a budget range per day based on their metabolic rate (as measured using a metabolic rate meter such as an indirect calorimeter), estimated activity levels, and weight control goals. The person can also be provided with a budget of diet dollars to be spent over the time of the program. This budget is preferably determined using metabolic rate measurements. Extra diet dollars can be earned through physical activity. An improved weight control program has no rigid requirement that the amount spent each day is the same. Deviations from planned expenditure are dealt with by re-budgeting, for example using a computing device such as a computing device. Days with high expected calorie intake, such as holidays, can be included into the program, and compensated for by planning lower calorie intakes during the other days of the program.

Towards the end of a weight control program, it can become impossible to reach a weight-loss or weight gain goal without exceeding a medically safe range of food intake. In this case, the computing device calculates revised goals based on a realistic diet and exercise regime.

Physical activity can be translated into weight control parameters, advantageously allowing a calorie balance between food intake, metabolism, and activity to be determined. The balance can be presented in terms of calories, diet points, or some other parameter.

The translation of physical activity into activity points can be based entirely on the calorie expenditure. The expended energy in kilocalories can divided by k₁ from Equation 1 to provide an expenditure point, or activity point, which can be balanced against diet parameters such as diet points. In this case, an activity resulting in a 100 kilocalorie energy expenditure will be equivalent to 5 activity points if k₁=20, and the person's diet point allowance can be increased by 5 diet points.

The metabolic rate of a person may remain higher than the resting rate after the activity has finished, falling over time if no further activity follows, and this additional calorie expenditure can be accounted for in the point equivalent of the activity.

More generally, the point equivalent of the exercises (activity points) will be the extra kilocalorie expenditure divided by a term k₄, where k₄ is a number correlated with k₁. The term k₄ may be a lower value for a certain amount of exercise energy expenditure per day, and then increase for additional activity, in order to encourage a certain minimum level of activity per day.

The term k₄ may also be calculated from an estimated (or recorded, target, or preferred) average fat and fiber content in the diet of the person in calculating activity points. For example, if a person typically consumes 12 grains of fat and 10 grams of fiber per 100 kilocalories of dietary intake, then the activity point count per 100 kilocalories is (using Equation 2):

$${P}_{A} = \frac{100}{20} + \frac{12}{12} - \frac{10}{5} = 4$$

If k₄ is calculated from this previous dietary intake, then in this case k₄=1.25×k₁=25. The value of k₄ will actually increase with a healthier diet (low fat, high fiber), which reduces the point credit of the exercise. It may be preferable to lower the value of k₄ by some additional multiplier e.g. 0.8, to increase the point credit for exercise. Alternatively, k₄ can be calculated using the fat and fiber consumption of a preferred diet, such as one consistent with dietary goals.

A person's resting energy expenditure and estimated activity level can be used to determine a number of diet points allowed over the time period of the weight control program. The number of allowed diet points can be related to the calorie expenditure due to metabolism, summed with the estimated activity energy, and then divided by some coefficient. The value of this coefficient will be related to k₁, and may depend on the planned diet components. Additional diet points can be allowed due to activity of the person.

In a system embodiment, a computing device, such as a personal digital assistant (PDA) is communication with a metabolic rate meter, such as an indirect calorimeter, and a body mounted activity sensor. This configuration allows the activity point value of an exercise to be determined. The portable computing device can also be in communication with an exercise machine, which can transmit activity level parameters, such as exercise intensity, repetitions, repetition rate, running speeds, physiological parameters (such as heart rate), metabolic expenditure, position location device (e.g. global positioning system data), and the like, to the portable computing device. The portable computing device may also communicate with other physiological sensors (e.g. blood glucose), or monitoring devices such as weight scales. The preferred methods of data communication to and from the portable computing device are wireless, such as the Bluetooth wireless communication protocol, IEEE802.11, IEEE802.11(b), wireless Ethernet, IR, and the like. However, cable links can also be used.

A portable computing device can also be in communication with a computer, interactive TV, or other electronic device with enhanced (relative to the portable computing device) display capabilities, so as to provide the person with a review of progress and feedback messages. The enhanced-display device can have a communications network (e.g. Internet) connection with a remote computer system. The portable computing device may also have a wireless connection to the remote computer. Data may be stored on the remote computer, for viewing by any authorized person (e.g. diet consultant, physician) by e.g. an Internet web page. Weight control data can also be transmitted to a remote computer system, for example using a wireless Internet connection or telephone link, and feedback received on another device such as an interactive television over a high speed data link.

In an improved weight loss method, a person carries a portable computing device, such as the Palm Pilot produced by Palm Computing. The metabolic rate of the person is measured using an indirect calorimeter, preferably the GEM (gas emission monitor) device. The metabolic rate is stored in the portable computing device, and converted into expenditure points (resting points). Food consumption is monitored using the portable computing device. The type of food eaten is entered through a menu-type interface, with the nutrition information retrieved from a database. Parameters, for example the points system of U.S. Pat. No. 6,040,531 (Miller-Kovach et al.) are calculated and stored in the portable computing device. The portable computing device is used to help plan future meals based on the food intake suggested by the weight control program.

Activity levels can be either estimated based on lifestyle, monitored using e.g. body-mounted accelerometers, or entered into the portable computing device e.g. using a menu-type system. For example, a person might enter “Activity—Walk—20 minutes” into the portable computing device through a menu type system. The portable computing device may also be used to time activities, or measure the length of walks, runs etc. using a global positioning system (GPS). The activity levels can converted to activity points by dividing the extra energy expenditure resulting from the activity, in kilocalories, by the constant k₁ used in the calculation of diet points (Equation 1).

If prepackaged meals are supplied as part of a weight control program, nutrition data for these meals can accessed by the portable computing device, e.g. using a database, and the person only has to identify the product using e.g. the product name, a bar code reader, a code (e.g. the universal product code, UPC) printed on the package, etc. for the nutrition information and/or diet points to be recorded. The database might be on the portable computing device, on a remote computer accessed via the Internet, or loaded onto the portable computing device via a memory module or data transfer from e.g. the Internet.

A portable computing device can be used to monitor the calorie balance and/or the balance of points for the person in a weight control program. The person is credited with a number of resting points per day based on their metabolic rate. The balance is debited by diet points related to the person's consumption. The balance is credited with activity points as a result of exercise. The person's metabolic rate is measured periodically, e.g. every two weeks, and the point credit per day modified if the resting energy expenditure changes. This improved weight control method encourages the person to increase or at least maintain their metabolic rate through activity. It is common for metabolic rate to fall while dieting, which undermines progress to a given weight goal. The improved weight control method helps avoid this problem by measuring metabolic rate.

The person's weight is measured at intervals, for example every week, and the data entered into the portable computing device by any convenient method. Significant discrepancies between the measured weight and the expected weight based on the recorded data on the portable computing device may result in e.g. the weight loss goals being revised, an appointment with a councilor, re-measurement of metabolic rate, etc.

The data stored on the portable computing device is transmitted via the Internet to a remote computer system, accessible by the person through the Internet, or by a diet councilor or other authorized person. Hence the person's weight loss program is conveniently monitored, and signs of problems allow an appointment with a councilor or physician to be made conveniently.

A portable computing device with a wireless Internet connection can also be used to order food from many sources, e.g. a company supplying weight loss products. The progress of the person towards the weight loss goal may be used to suggest product orders.

A business model for the administration of a weight loss program can comprise the following elements. A weight loss company supplies a customer with a portable computing device, such as a Palm Pilot, or appropriate software if the customer already owns a suitable device. The customer pays the weight loss company a monthly fee for participation in the program. An extra charge can be made for the portable computing device, or this may be waived if the customer signs up for a certain minimum time period. The metabolic rate of the person is measured using an indirect calorimeter, which can be in possession of a local representative of the weight loss company. The customer may be supplied with his or her own GEM for an extra fee or rental charge. Based on the metabolic rate of the person, expected activity level, and desired weight loss goal, an average food intake value is calculated, in terms of calories or other diet parameters such as diet points. The customer uses the portable computing device to record dietary information and activity levels. The customer has access to a representative of the weight loss program, either a local representative or through the Internet, who can be supplied with all recorded data. Counseling can be supplied based on the information recorded on the portable computing device and progress towards the goals. A computer expert system can be used to provide advice.

The weight loss company can further supply the customer, on demand, with prepackaged meals. The nutrition information for such meals can be downloaded once onto the computing device via the Internet, then stored for future use, or supplied in any convenient way (e.g. via transfer of a memory module). Product identifiers can be entered into the computing device using a software application program (for example using a menu system), a bar code scanner, entering numeric codes, and the like.

An improved diet control method is now described. A person with a medical disorder such as diabetes has to monitor their diet very carefully. Many such people use the Exchange Lists for Meal Planning developed by the American Diabetes Association to plan meals. It is difficult to keep track of the exchange equivalents of meals, and even more difficult to integrate this system with an exercise program.

The person carries a portable computing device, which stores the exchange values (e.g. starch, fruit, fat, etc.) for various food items. An average daily calorie intake is devised for weight maintenance (i.e. weight stability). The portable computing device is used to plan meals based on the required number of exchanges per day and calorie intake, and to keep track of food consumption. Activity points are added if certain exercises are performed, converted into fractional exchanges by the portable computing device, and used in diet planning.

Preferably, food consumption is recorded at the time the food is eaten, or the time of food consumption recorded if the data entry -is made later. The response of the person's blood sugar level is either tracked using glucose sensors (which might be build into the portable computing device), or predicted using models based on previous measurements of that person's physiology. The blood sugar response, and it's predicted future behavior, can be used to suggest future meals, eating times, and appropriate exchange contents. The portable computing device is also used to recommend safe times for exercise, e.g. when blood sugar levels are not at risk from falling outside safe levels.

A diet control method with an exercise component is now described. A person monitors their calorie intake using a portable computing device. In addition, they engage in exercises, for example walking, running, and the like so as to expend calories. The American College of Sports Medicine (ACSM) has produced metabolic equations to calculate the energy expended by various forms of exercise. Using the equation in the form given by David P. Swain and Brian C. Leutholtz in their book Metabolic Calculations—Simplified (Williams and Wilkins, 1997) the equation for oxygen consumption for a person walking is

VO₂=3.5+2.68 s+0.48 sg (Equation 3)

where VO₂ is the oxygen consumption rate per unit body mass in ml.min⁻¹.kg⁻¹, s is the walking speed in mph, and g is the grade in percentage (i.e. g=5 for a 5% grade). VO₂ can be converted to energy expenditure in kcal.min by multiplying by the person's body weight in kg, and dividing by 200. The term 3.5 in Equation 3 is related to the average resting energy expenditure per unit mass of the population. The two right hand terms are related to the extra energy expenditure due to the exercise.

In the improved weight loss program, an indirect calorimeter is used to determine the person's resting oxygen consumption rate per unit mass. This measurement gives a more accurate number than the constant term 3.5 in Equation 3. The GEM can also be used to measure VO₂ during exercise to obtain more accurate parameters for the activity-related energy expenditure terms in Equation 2. However, since the resting metabolism is the most important term in calculating energy expenditure over the course of a day, the person can use the resting energy expenditure measured by an indirect calorimeter, but the standard ACSM equations to calculate the extra calories burned during the weight loss program.

For example, a 100 kg person walks at 3 mph on a treadmill at a 5% grade for 20 minutes. The additional kilocalories ΔC burned, over the resting metabolism, is equal to (using Equation 3):

$$Δ C = ( 2.68 \times 3 + 0.48 \times 3 \times 5 ) ( 20 \times \frac{100}{200} ) = 152 kcal$$

The time period corresponding to a single point expenditure can be determined, and used to calibrate an activity signal from an activity monitor into points per unit time.

If the person is using the a diet points system using Equation 1, this value of ΔC, the activity energy expenditure for the exercise, can be converted into points by dividing by a number (k₄) approximately equal to k₁. Using the numerical values of Equation 2, k₁=20. However, to encourage exercise, k₄ can be a lower value, such as 18, so that the person receives 8 points (to the nearest integer). A portable computing device can be used to carry out these calculations.

The following example illustrates an improved weight loss program with an activity component. The person has their rest metabolic rate measured using an indirect calorimeter, such as the GEM (gas exchange monitor) invented by James Mault. The GEM measures rate of oxygen consumption, from which metabolic rate is calculated. For user convenience, the indirect calorimeter can also calculates the number of diet points per day that the person should consume, based on the measured resting energy expenditure (or oxygen consumption). This number can be increased based on an estimated activity level, and can be stored on the portable computing device and used in diet planning. The GEM can also be modified by adding a carbon dioxide sensor (e.g. using a solid state IR source and detector) for respiratory quotient measurements. The respiratory quotient is a measure of carbon dioxide production per unit volume of oxygen consumed, and knowledge of this number allows a more accurate estimated of metabolic rate to be made.

The act of diet control can affect the resting metabolism in unpredictable ways, which is a known problem in implementing diet programs. Use of the GEM at regular intervals, e.g. bi-weekly, allows the diet-plan to be compensated for changing rest metabolism. Another important advantage of monitoring the metabolic rate is that it encourages activity by the person. Conventional diet plans may leave a person feeling low on energy, and the rewards (if any) for activity may not be sufficient to motivate the person to exercise. However, if a person can raise their resting energy expenditure by exercising, the improved diet control method allows for the food allowance to be increased. An increased resting metabolism can also indicate the conversion of fat mass to muscle mass, which is a health-beneficial outcome of a diet and exercise program not monitored by the simple body weight measurements of conventional programs.

Other embodiments of the invention are possible, for example combining selected elements of the above examples. The examples emphasize weight loss, as this is of interest to most people on a diet program, but weight maintenance and weight gain programs are also possible.

Hence, a process for controlling body weight of a person over a period of time, comprising the steps of measuring the metabolic rate of the person, using the metabolic rate of the person to determine the food intake requirements of the human over the period of time, adding to the food intake requirements based on the activity of the person, using an electronic device to monitor food intake over the period of time, and using the monitored actual food intake and physical activity to calculate a revised suggested food intake for the remaining portion of the period of time, whereby the human is assisted in reaching a weight control goal.

Embodiments of the present invention can be used in relation to other mammals, for example, horses. An indirect calorimeter can be provided with a horse mask, and used to determine the resting points and activity points corresponding to a horse at rest and exercising, respectively. Diet points can be assigned to horse feed based on calorie content and other horse nutrient content.

Hence, a method of assisting a person to achieve a weight control goal comprises the determination of a resting energy expenditure for the person using a metabolic rate meter such as an indirect calorimeter; and assigning resting points to the resting energy expenditure of the person. Activity points are assigned to an activity level of the person. Consumption of consumables (such as food, beverages) by the person is monitored, and consumption points (such as diet points) are assigned to the consumables consumed, wherein the consumption points are assigned based on the calorie content and at least one other nutritional parameter of the consumables such as the content of fat, fiber, minerals, vitamins, cholesterol, lipids, protein, carbohydrate, complex carbohydrates, sugars, glycemic index, and the like. The difference between the consumption points and a summation of the expenditure points with the activity points is then determined; and feedback to the person is provided based on the difference. The activity points and/or resting points can be assigned based on the caloric value of the respective energy expenditure, and estimated, assumed, measured, or otherwise determined content of nutrients in the person's diet used in the calculation of consumption points, as described in more detail below, so as to be consistent in magnitude with diet points. The activity level of the person can be determined using a portable activity monitor such as a pedometer, physiological monitor, and the like.

A system for assisting a person in a weight control program comprises an activity monitor having an activity sensor and a transmitter, wherein the activity monitor transmits an activity signal correlated with a physical activity level of the person; a portable electronic device having a processor, a display, a receiver, and a memory, wherein the portable electronic device receives the activity signal from the activity monitor; and a software application program running on the portable electronic device, adapted to receive data corresponding to consumables consumed by the person, to assign diet points to the consumables consumed based on calorie content and at least one other nutritional parameter, to assign activity points correlated with the activity signal, to receive a resting energy expenditure of the person, to assign resting points correlated with the resting energy expenditure, and to provide a visual representation of the difference between the diet points and the sum of the resting points and the activity points on the display.

A method of assisting a person t0 achieve a level of physical activity comprises: determining a resting energy expenditure of the person; assigning a number of activity points to the calorie value of the resting energy expenditure, so that a single activity point is a fraction of the person's resting energy expenditure; monitoring an activity level of the person; correlating the activity level of the person with a value of activity points expended; and providing feedback to the person correlated with the value of activity points expended. The resting energy expenditure of the person can be determined using an indirect calorimeter. The Harris-Benedict equation can also be used, as is known in the art. The feedback can comprise a graphic display on an electronic display, such as a bar chart, the illumination of colored lights such as light emitting diodes, which may form a bar-graph, an audio signal, a numeric display, and alphanumeric display, voice synthesis, vibration of the activity sensor, flashing light, wavelength-changing lights (such as multi-color, and the like.

A system to assist a person achieve a level of energy expenditure comprises: an activity sensor; a processor, receiving an activity signal from the activity sensor, adapted to determine an energy expenditure by the person from the activity signal, and to generate a visual representation on a visual indicator of a predetermined range of energy expenditure within which the energy expenditure falls. The predetermined range of energy expenditure can be calculated based on the resting energy expenditure of the person, such as a range forming a fraction of REE. The predetermined range can also be a fixed numeric range of calorie or activity point expenditure. The visual indicator can be a electronic display, a plurality of colored lights such as light emitting diodes, a bar graph, and the like. The activity sensor can be a heart rate sensor, an accelerometer, a pedometer, a position location system such as global positioning system, an indirect calorimeter, or a repetition, weight, or other sensor forming part of an exercise machine.

The entire contents of the following are incorporated herein by reference: U.S. provisional application Serial Nos. 60/207,089, filed May 25, 2000; 60/225,101, filed Aug. 14, 2000; U.S. patent applications Ser. Nos. 09/630,398, filed Aug. 2, 2000, 09/684,440, filed Oct. 10, 2000; and 09/745,373, filed Dec. 23, 2000; U.S. Pat. Nos. 6,135,107, 5,836,300, 5,179,958, 5,178,155, 5,038,792, and 4,917,108, and International applications Nos. WO 00/07498A1, published Feb. 17, 2000 and WO 01/08554A1, published Feb. 8, 2001.

### BRIEF DESCRIPTION OF THE DRAWINGS

FIG. 1 shows a schematic of a calorie management system.

FIG. 2 shows a schematic of a calorie management system.

FIG. 3 shows a schematic of a portable device for use in a calorie management system.

FIGS. 4A-4E show a flow chart corresponding to a calorie management software program.

FIG. 5 shows a person walking on a treadmill while breathing through an indirect calorimeter.

FIGS. 6A and 6B show an indirect calorimeter of a type, which can be advantageously used in systems according to the present invention, more fully described in a co-pending application to Mault et al.

FIG. 7 show a cross-section of the indirect calorimeter shown in FIG. 6.

FIG. 8 shows a flow chart for calibrating activity level against metabolic rate using an indirect calorimeter.

FIG. 9 shows a system for providing feedback to a person regarding calorie balance.

FIG. 10 illustrates how activity zones can be defined.

FIG. 11 illustrates a method of defining activity zones.

FIG. 12 represents a person (lower half only shown) performing an exercise while receiving feedback based on activity points.

FIG. 13 shows a schematic for a feedback device using activity points.

FIG. 14 shows a method of calibrating an activity monitor by performing one activity point (or some other known value) of exercise.

FIG. 15 shows a system embodiment according to the present invention.

### DETAILED DESCRIPTION OF THE INVENTIONS

Methods of Calculating Activity Points

Calorie expenditure is the sum of resting metabolism, or resting energy expenditure (REE) and activity energy expenditure (AEE), which together form the person's total energy expenditure TEE, i.e.:

TEE=REE+AEE (Equation 4)

Conventionally, TEE and REE are given in terms of calories per day. Other time periods can be used if appropriate. In equation 4, AEE represents the calorie value of exercise per day. However, AEE can also used to represent the energy expenditure during a particular activity.

TEE is a better guide to a person's calorie intake needs than body weight. Hence, in an improved weight control program, a person's calorie requirements or diet point allowance can be determined from a measurement of REE using, for example, an indirect calorimeter, and a measurement or estimate of AEE.

If a person is recording diet points using an equation disclosed in Miller-Kovach et al, such as Equation 1 discussed above, namely:

$${P}_{D} = \frac{c}{{k}_{1}} + \frac{f}{{k}_{2}} - \frac{r}{{k}_{3}} \qquad ( Equation 1 )$$

then REE can be used to determine a resting points allowance. A simple relationship can be used, such as Equation 5 below, which gives a daily resting point allowance PR as:

$${p}_{R} = \frac{REE}{{k}_{1}} \qquad ( Equation 5 )$$

The daily resting point allowance P_{R} can also be calculated using the following equation (Equation 6):

$${P}_{R} = \frac{REE}{{k}_{1}} + \frac{f}{{k}_{2}} - \frac{r}{{k}_{3}} \qquad ( Equation 6 )$$

where f and r are the person's total fat and fiber allowance per day, based on the calorie value of TEE, and the recommended dietary proportion of fat and fiber for this calorie value of consumption. The value of the terms f and r can also be determined using the person's actual fat and fiber consumption for a given calorie consumption, as recorded in a diet log.

Referring to Equation 6, for example, if REE=2,000 kcal, f=72, r=20, k₁=20, k₂=12, and k₃=5, then the person's daily resting point allowance P_{R} will be given by (using Equation 6):

$$\begin{matrix}{p}_{R} = \frac{2000}{20} + \frac{72}{12} - \frac{20}{5} \\ = ( 100 + 6 - 4 ) points \\ = 102 points .\end{matrix}$$

(If forms of Equation 1 and Equation 6 are used in which the fiber term is not used, then the daily allowance of points will be (100+6) or 106 points, using the same numbers as the example given above).

Alternatively, the person's daily resting point allowance P_{R} can be defined by an equation:

$${P}_{R} = \frac{REE}{{k}_{5}} \qquad ( Equation 7 )$$

where k₅ is chosen to give a number of diet points more or less than that number best corresponding to resting energy expenditure, so as to lead to weight gain or weight loss respectively for the person.

Using Equation 6 reduces the resting points corresponding resting energy expenditure available when a healthy diet is planned having low fat and high fiber content. Hence, Equation 7 may provide advantages. However, the use of equations such as Equations 5, 6, and 7 provides a great advantage in that they define resting points, which can be compared with diet points, allowing a calorie balance to be conveniently determined in diet points.

Expenditure points can also be calculated for activities such as exercise programs. Tables and formulae are known which provide calorie expenditure estimates for common exercises. These activity caloric expenditures can be converted into activity points. An indirect calorimeter can be used to determine energy expenditure during exercise, as described in more detail below.

A value of AEE, determined using any method, can be converted into expenditure points (activity points) using the same methods described above for REE. For example, Equation 8 (analogous to Equation 5) allows calculation of activity points P_{A}.

$${P}_{A} = \frac{AEE}{{k}_{5}} \qquad ( Equation 8 )$$

Equation 9 below, for calculation of activity points, is analogous to Equation 6.

$${P}_{A} = \frac{AEE}{{k}_{1}} + \frac{f}{{k}_{2}} - \frac{r}{{k}_{3}} \qquad ( Equation 9 )$$

However, exercise is known to be highly beneficial in health maintenance programs, weight control programs, and the like. Hence, a formula of the type given below can be used to calculated activity points so as to encourage activity:

$${P}_{A} = \frac{AEE}{{k}_{4}} \qquad ( Equation 10 )$$

where k₄ is chosen to encourage exercise, for example through being 5%-50% lower than k₁. The value of k₄ can be the same as k₅ in Equation 8.

Calorie Balance in Points

For weight loss, diet points consumed must translate to a lower calorie intake than the total energy expenditure. A weight loss goal can be achieved by allowing a person a lower level of diet points than corresponding calorie expenditure points (the sum of activity points and resting points). For example, after determining the total energy expenditure of a person, the diet point allowance can be set to be 5% less than the sum of activity points and resting points. Alternatively, activity points and diet points can have slightly different scales. For example, a diet point can correspond to a smaller number of calories than a calorie expenditure point. The rate of exchange, for example, can be that the diet point corresponds to 5% less calories than an activity point. The rate of exchange between calorie expenditure points and diet points then controls the rate of weight loss.

Hence, having determined daily totals for P_{A} and P_{R}, a daily diet point allowance can be determined by summing P_{A} and P_{R}, then adding or subtracting an adjustment factor based on weight loss goals. The value of this adjustment factor is determined by a calorie density term relating weight changes to calorie deficit or surpluses, as is well known in the art. The point balance between P_{D} (cumulative for consumed items) and the sum of P_{A} and P_{R} can be shown to the person as a visual representation on the display of an electronic device, for example as an numeric value, graphic, alphanumeric display, bar graph, and the like. The device can show the balance in directly in points, without revealing details of bow activity points, resting points, and, diet points are calculated. For simplicity, the values of each of these weight control parameters can be presented to the person as “points”, without further descriptive labels.

If TEE for the person is determined, for example by measuring REE using an indirect calorimeter, and estimating or determining AEE, then a diet point (P_{D}) allowance can be determined for a person by substituting TEE in place of REE in equations 5, 6, or 7. For example:

$${P}_{D} = \frac{TEE}{{k}_{1}} \qquad ( Equation 11 )$$

The value of P_{D} determined using such equations can also be adjusted to be consistent with planned weight gain or weight loss goals.

In general, a diet point equation containing a calorie term (such as a calorie value divided by some constant) and at least one other term corresponding to a nutritional component can be modified (by analogy to the above methods) to define resting points, activity points, or diet points based on total energy expenditure. The energy expenditure due to the resting metabolism and/or activity is entered as the calorie value term, and other nutritional parameters are entered according to actual, planned, or healthy levels of the nutritional parameters for that calorie value of food consumption. The number of diet points allowed for a given weight control goal is then calculated.

By completing exercise and activity programs, a person can be credited with activity points, and correspondingly increase their diet points allowance, allowing them to eat more during the day while adhering to a weight control program. For example, a person walks for an hour, expending 160 kcal. This calorie value can be converted to activity points using an equation such as those given above. For example, if Equation 2 is used to calculate diet points, using Equation 8 gives a value of 8 activity points for the walking. These activity points can be added to the person's diet point allowance, based on resting metabolism and any additional diet point allowance related to conventional lifestyle energy expenditure, increasing the person's allowance of diet points for food consumption.

For a typical person, approximately 70% of TEE is related to resting metabolism. A common failing of conventional weight control programs is the failure to determine REE, and account for variations in REE during the course of the weight control program. REE is conveniently and accurately determined using an indirect calorimeter, for example, devices as described by James R. Mault, M. D. and others, for example in U.S. Pat. Nos. 6,135,107, 5,836,300, 5,179,958, 5,178,155, 5,038,792, 4,917,108 and published international applications WO001/08554 and WO000/07498, the contents of all of which are incorporated herein by reference.

Further, an improved method for determining diet points allowance for a person comprises: determining REE using an indirect calorimeter; estimating AEE from lifestyle details; determining TEE from the sum of REE and AEE; and determining the total allowable diet points from TEE. For example, if REE is determined to be 1,800 kcal, AEE can be estimated to be a certain fraction of that value, for example based on the person's waking hours, nature of employment, exercise levels, and the like.

Weight Control System

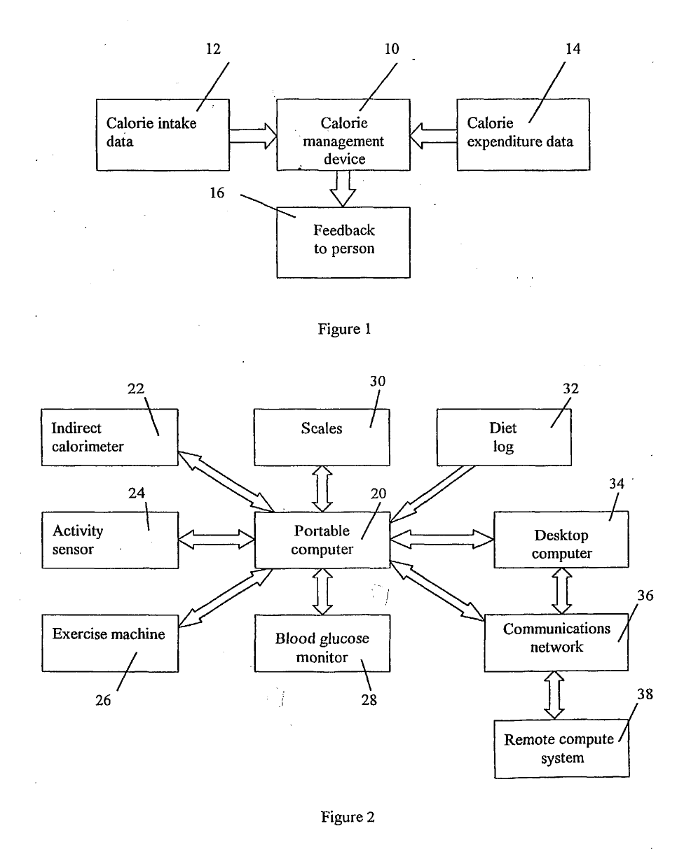

FIG. 1 schematically illustrates a calorie management system. A calorie management device (10) receives data corresponding to the calorie intake (12) and calorie expenditure (14) of a person, and provides feedback (16) to the person based on the comparison. For example, calorie intake can be monitored using diet logging software on the computing device. Calorie expenditure (TEE) can be determined from measurements of a person's resting energy expenditure (REE) and activity energy expenditure (AEE). Feedback can be in the form of suggestions or advice, e.g. modified meals, exercise programs, etc.

The calorie management device is preferably a portable computing device, such as a personal digital assistant (PDA), for example Palm, Handspring, and PocketPC models. The calorie management-device can be any computing device or portable electronic device with additional functionality, such as a calculator, computer, pager, wireless phone, and the like. For convenience, the calorie management device will be referred to as a computing device.

A person can record diet points corresponding to food eaten using software on the computing device 10. These points may be marked on packaged foods, such as foods supplied by a weight management business. Points may also be provided by lists, tables, and the like, or can be calculated from data given as calories. In a preferred embodiment, a portable computing device is used to record food eaten, activities performed, and resting energy expenditure, to determine the corresponding diet points and calorie expenditure points, and to present a calorie balance to the used in terms of points.

FIG. 2 shows a weight control system according to embodiments of the present invention. The system comprises a portable computing device 20, an indirect calorimeter 22, an activity sensor 24, an exercise machine 26, a blood glucose meter 28, scales 30, a diet log mechanism 32, a communications link to a desktop computer system 34, a communications network 36, and a remote computer system 38. The portable computing device 20 is preferably a PDA (personal digital assistant) such as a Palm PDA, pocket PC, and the like.

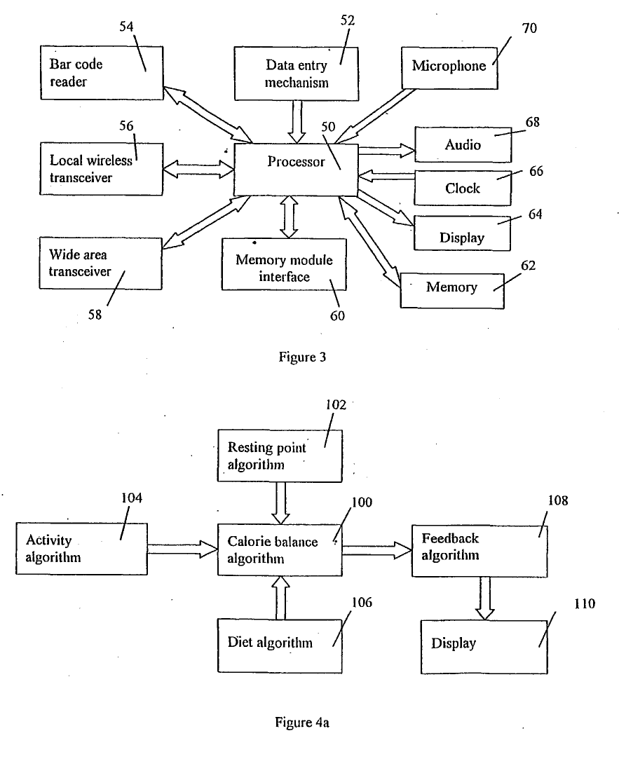

FIG. 3 shows a schematic diagram of a portable computing device that can be used in the present system. The computing device comprises a processor 50, a data entry mechanism such as a keyboard, stylus, and the like 52, a bar code reader 54, a local wireless transceiver 56, a wireless transceiver 58, a memory module interface 60, a memory 62, a display 64, a clock 66, an audio output device such as a speaker 68, and a microphone 70. The barcode reader 54 can be used for reading data off of packaged materials, such as UPC codes, and also for reading data from exercise machines suitably labeled. The local wireless transceiver 56 is preferably a low power Bluetooth transmission/receiving system for receiving data from sensors on the body, such as activity sensors. The wide area wireless transceiver 58 provides access to a communications network such as the Internet, or wireless phone functionality. The memory module interface 60 provides the ability to read or write data to or from memory modules, such as flash memory, memory sticks and the like. This data may include data recorded by physiological sensors. The memory 62 can comprise conventional RAM or ROM memory. The display 64 can be used to provide visual representations of diet points consumed and expenditure points expended, and can be any conventional display. The audio output device 68 can be used to provide feedback to the person, for example alerts for exercise times. The microphone 70 can be used as part of a voice recognition system for entering data into the computing device. Referring back to FIG. 2, the computing device 20 has a software application program adapted to monitor the calorie balance of the person. At intervals, the person measures their resting energy expenditure using the indirect calorimeter 22. The determined value is entered into the PDA by any convenient method and stored in the memory. The determined value of REE can be used to determine a number of resting points for the person.

The person carries an activity sensor 24, which provides a signal correlated with the physical activity level of the person. This can be a body mounted accelerometer, such as one providing an electrical signal correlated with the vertical component of acceleration of the person's torso. The activity sensor can be a pedometer, providing a signal correlated with the number of steps, paces, or other repetition of an exercise routine. The activity sensor may further be a heart rate sensor, or other physiological sensor.

At intervals, the person measures their body weight using the scales, and enters their body weight into the PDA. Changes in body weight can be correlated with the calorie balance of the person. It may also be correlated with the hydration level of the person as determined using bioimpedance measurements.

The person can also carry a blood glucose meter, which transmits blood glucose values at intervals to the PDA. This can be used in meal planning and other purposes.

The person can also at intervals, use an exercise machine. The type, repetition number, and intensity (or activity level) of exercise performed can be transmitted to the computing device. For example, low power wireless transmission, manual entry of data, barcode scanning, and any other convenient methods can be used.

Software for Calorie Balance

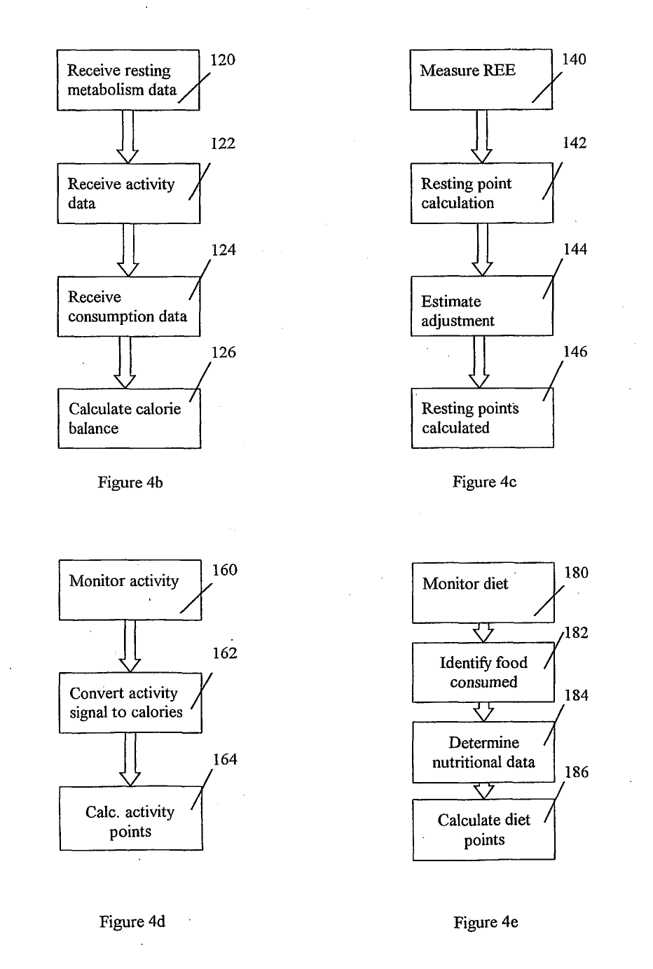

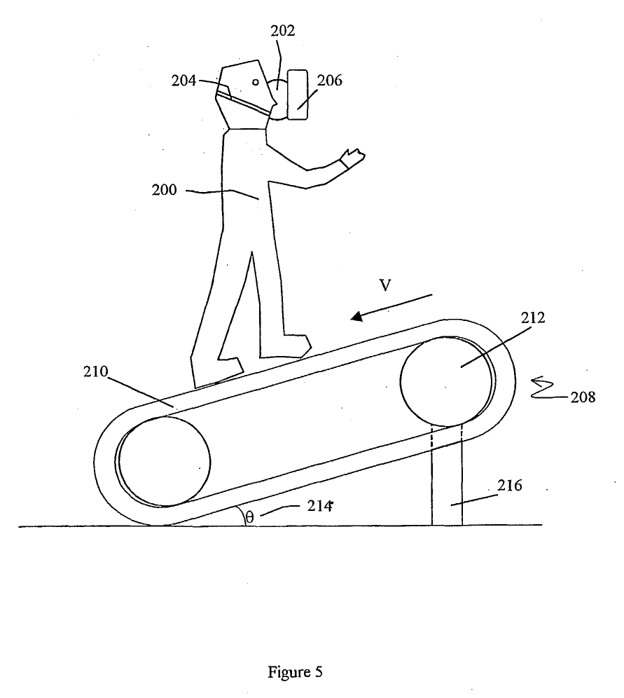

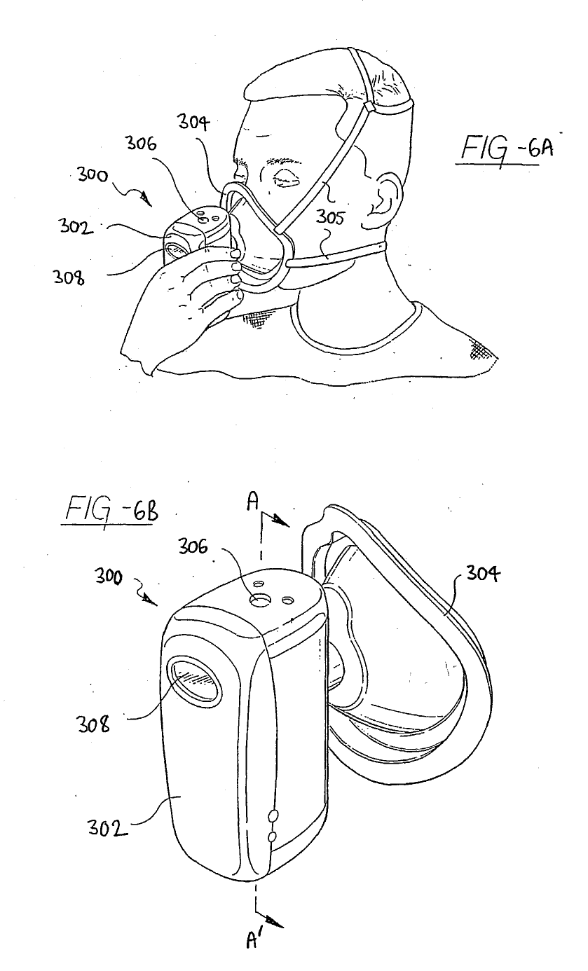

FIG. 4a shows a schematic of a software program for providing a person with feedback based on their calorie balance. The calorie balance algorithm 100 is shown in more detail in FIG. 4b. The resting point allowance is received from the resting point algorithm (102 and FIG. 4c). At intervals the person measures their resting metabolism. For example, a person can be prompted every week or more frequently at the start of a diet program. The measured REE is converted into a number of resting points using methods discussed above.

The calorie balance algorithm receives the number of resting points (120), adds the activity points (122), subtracts the diet points corresponding to the person's consumption (124), and generates the calorie balance in points for display to the user.

The activity monitor algorithm (104, FIG. 4d) receives data from an activity monitor such as a body mounted accelerometer (160). The activity signal is converted into an activity expenditure energy in calories (AEE) 162, and/or to activity points (164).

The diet log algorithm (106, FIG. 4e) receives information on food consumed, for example by manual entry of product codes, barcode scanning, entry of UPC codes, and the like. The consumed food codes can then be converted to food identities, and hence the nutritional information. If a person is eating prepackaged foods supplied by a weight loss company, the product codes can be readily entered into the diet log software. Packages can also be labeled with transponders or transmitters acting as enhanced barcodes, and providing additional information such as calorie and nutritional content. Box 180 corresponds to diet monitoring, for example using a diet log program, box 182 corresponds to identification of foods from product identifiers such as bar codes, names, or numeric codes, box 184 corresponds to correlation of product identity with nutritional data (for example using a database on a computing device), and box 186 corresponds to the determination of diet points from the nutritional data.

The calorie balance algorithm (100, FIG. 4b) calculates calorie balance on a daily basis. The balance can be used to help the person plan meals and activities. For example, the person can be presented with a list of meals based on a current balance, and additional options presented on the condition that one or more activity points of exercise are completed.

The use of points rather than calories allows a person to be presented with conveniently scaled numbers, which can be easily converted to food or activity equivalents.

Activity Monitoring

The conversion of the activity monitor signal to activity points can be assisted by a portable computing device. For example, the signal from a pedometer can be converted using a factor accounting for the energy expenditure of the person per mile walked. Such conversion factors are known in the exercise arts. In place of an activity monitor, the person can use a simple timer to measure the duration of an activity, and this duration can be converted to an energy expenditure value. A person can also enter the time, duration, intensity, and repetitions (if appropriate) of an exercise into a software program running on the portable computing device. For example, a menu system can be used for data entry.

The PDA can also be used to guide an exercise program. For example, suppose a person receives one exercise point per fourteen minutes of walking. The portable computing device, or timing device, can be used to sound an alarm every fourteen minutes, and a distinct alarm after a predetermined target has been reached.

For exercises with a number of parameters, the parameters can be entered into the PDA and used to calculate points per minute. An example is treadmill work, in which gradient and treadmill speed are the variable parameters. An alarm can sound after each point is achieved.

Indirect Calorimeter Provided Point Reading

The output from an indirect calorimeter can be presented to the user in terms of calories per day, or equivalent activity points as defined using the methods above. The conversion from calories to points can be carried out automatically by the indirect calorimeter, for example by assigning a certain number of kilocalories per point, by converting using Equations 5,6, or 7, or another appropriate equation. An assumed fat and fiber consumption per day can be used when evaluating points using Equation 3.

Alternatively, the metabolic rate reading from the indirect calorimeter can be transmitted to or manually entered into a portable computing device, and the conversion from REE in kilocalories per day to activity points can be achieved using software running on the portable computing device. The conversion can use any convenient approach.

Calibration of Activity Monitors

A number of devices may be used to generate a signal proportional to physical activity level. These include: body mounted accelerators; posture sensors, e.g. ultrasonic distance sensors; pedometers; GPS (global positioning system) or other positioning equipment or methods; muscle activity sensors; physiological sensors (e.g. heartbeat, respiration rate, skin conductivity, skin temperature, blood flow, chest expansion due to air intake); exercise machines, which can provide a signal related to the number and difficulty of exercises performed; and indirect calorimeters, which may measure VO2 (volume of oxygen consumed) and sometimes VCO₂ (volume of carbon dioxide exhaled), from which metabolic rate can be calculated.

In addition, formulas exist for calculation of the energy expended in various exercises, e.g. walking on treadmills, which are familiar to exercise scientists. However, these formulas tend to be for an average person, and do not take account of individual differences.

The inventor, James R. Mault, has invented an improved indirect calorimeter useful for measuring metabolic rate. A person at rest breaths into the indirect calorimeter through a mouthpiece, and their resting energy expenditure is determined. The person can then perform an exercise while wearing a mask connected via an air passage to the indirect calorimeter. The person's oxygen consumption can be determined while the person is exercising, and an accurate determination of the person's energy expenditure during the exercise can be obtained. Indirect calorimetry is a very accurate method of measuring the energy expended during an exercise or activity.

In a practical calorie management system, activity needs to be monitored over extensive periods of time, so that activity sensors must be unobtrusive. The indirect calorimeter is accurate, but not unobtrusive. Activity monitors can be calibrated against an indirect calorimeter, allowing an unobtrusive, inexpensive activity monitor to give a more accurate estimate of the person's energy expenditure rate.

In an illustrative example, the person carries a body mounted accelerometer, for example attached to a belt. This device will be referred to-as an activity monitor. Tile activity monitor provides a signal proportional to physical activity, for example the vertical components of body acceleration. In a preferred embodiment, the person also carries a portable electronic device such as a portable computing device, portable computer, wireless phone, electronic organizer, electronic book, etc. for the purpose of diet logging and receiving feedback. In another embodiment, the activity monitor and the portable computing device are the same device.

Preferably, the activity monitor communicates activity data to the portable computing device. The portable computing device is also used as a diet logger, allowing the portable computing device to calculate calorie balance and provide feedback to the person, e.g. concerning meal suggestions, exercise suggestions, etc. The preferred method of data transfer uses the Bluetooth wireless communication protocol. Other methods may include wires and cables, optical data transfer, transfer of nonvolatile memory cards, a physical connection, IR, ultrasound, etc. The portable computing device may also receive data from other physiological sensors or transducers carried by the person.

The person carries a portable computing device with an activity monitor mounted on a belt. In other embodiments, the activity monitor and GEM may be combined into the same device, for example recording activity when clipped to a belt and acting as a portable computing device when hand held. In order to avoid false signals, the activity monitor may detect when it is in an automobile, elevator, etc., e.g. by communication with various transmitters, or by recognizing the pattern of false signals.

Having obtained an activity signal related to the physical activity level of the person, the activity signal is converted into an energy expenditure by the person, in terms of calories or activity points. A preferred method of calibrating the activity monitor is using a metabolic rate meter such as an indirect calorimeter. A resting energy expenditure can be determined by having the person breathe into the mouthpiece while at rest. However, the device is also ideal for measuring the enhanced metabolic rate during activity/exercise. The GEM can be used with a mask, into which the person breathes while exercising.

Hence an indirect calorimeter is an ideal device for calibrating an activity monitor. The portable computing device or other device may prompt a series of exercises by the person. For example, the person might be requested to jog on the spot for five minutes, walk around for five minutes, run on the spot for two minutes, etc. The output from the activity monitor is monitored and correlated with data from the indirect calorimeter. Calibration data is therefore obtained for the activity monitor for these activities. A beep or spoken instruction may be used to indicate the change between each activity.

Having calibrated an activity sensor, the activity signal can then be converted to and displayed as points, either by the activity sensor itself (which can have a display or other visual indication of activity points expended), or other electronic device in communication with the activity monitor.

Activity Points During Exercise

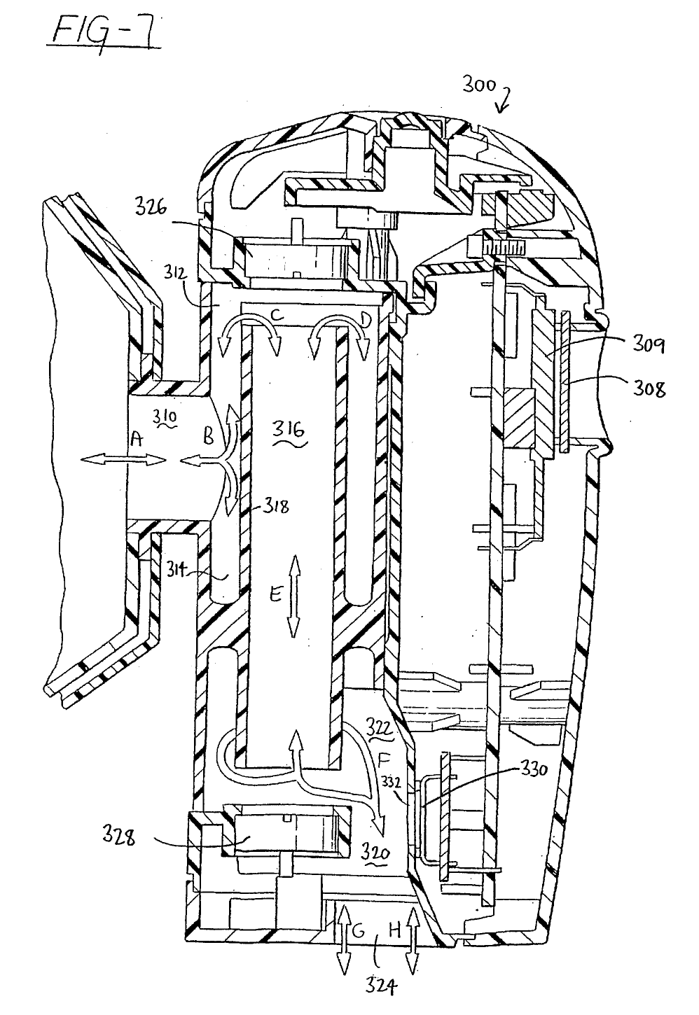

FIG. 5 shows a person 200 breathing into a mask 202 of indirect calorimeter 206 as the person walks on a treadmill 208. A strap 204 secures the mask around the head of the person.

The treadmill 208 comprises a conveyor belt 210, driven at a speed V by the drive wheel 212, and a gradient angle θ (214), varied by the height adjustment 216. The energy per unit time expended by the person 200 is measured using the indirect calorimeter. The energy expenditure in excess of resting energy expenditure, corresponding to AEE, can hence be determined and converted into points per unit time. Hence, in future use of the treadmill under similar conditions, the person need not use the calorimeter to determine energy expenditure. The person can use the determined calibration of points per unit time.

Gas Exchange Monitor (GEM)

FIGS. 6A and 6B show in more detail the person wearing a mask connected to the Gas Exchange Monitor (GEM), an indirect calorimeter developed by James R. Mault M.D. and others. Referring to FIGS. 6A and 6B, the calorimeter according to U.S. application Ser. No. 09/630,398 is generally shown at 300. The calorimeter 300 includes a body 302 and a respiratory connector, such as mask 304, extending from the body 302. In use, the body 302 is grasped in the hand of a user and the mask 304 is brought into contact with the user's face so as to surround their mouth and nose, as best shown in FIG. 6A. Optional straps 305 are also shown in FIG. 6A. With the mask 304 in contact with their face, the user breathes normally through the calorimeter 300 for a period of time. The calorimeter 300 measures a variety of factors and calculates one or more respiratory parameters, such as oxygen consumption and metabolic rate. A power button 306 is located on the top side of the calorimeter 300 and allows the user to control the calorimeter's functions. A display screen is disposed behind lens 308 on the side of the calorimeter body 302 opposite the mask 304. Test results are displayed on the display following a test. Other respiratory connectors can be used, for example a mouthpiece.

FIG. 7 shows a cross section of an indirect calorimeter, which can be used in embodiments of the present invention. The indirect calorimeter is best described in U.S. application Ser. No. 09/630,398, incorporated herein by reference. FIG. 7 shows a vertical cross section of the calorimeter 300, along section line A-A′ of FIG. 6B. The flow path for respiration gases through the calorimeter 300 is illustrated by arrows A-H. In use, when a user exhales, their exhalation passes through the mask 304, through the calorimeter 300, and out to ambient air. Upon inhalation, ambient air is drawn into and through the calorimeter and through the respiratory connector to the user.

Exhaled air passes through inlet conduit 310, and enters connected concentric chamber 312. Excess moisture in a user's exhalations tends to drop out of the exhalation flow and fall to the lower end of the concentric chamber 314. Concentric chamber 312 serves to introduce the respiration gases to the flow path 316 from all radial directions as evenly as possible. Exhaled air flows downwardly through a flow path 316 formed by the inside surface of the flow tube 318. Exhaled air enters outlet flow passage 320, via concentric chamber 322, and passes through the grill 324 to ambient air.

Flow rates through the flow path 316 are determined using a pair of ultrasonic transducers 326 and 328. An oxygen sensor 330, in contact with respiratory gas flow through opening 332, is used to measure the partial pressure of oxygen in the gas flow. Integration of oxygen concentration and flow rate allows inhaled oxygen volume and exhaled oxygen volume to be determined. The metabolic rate of the user is determined from the net oxygen consumption; the difference between inhaled and exhaled oxygen volumes. Metabolic rate is determined using either a measured or assumed respiratory quotient (the ratio of oxygen consumption to carbon dioxide production). For a user at rest, the REE (resting energy expenditure) is determined. The REE value is shown on display 309, behind window 308. Alternatively, VO2 can be displayed, from which REE can be determined using the Weir equation, as is well known in the art.

Preferably, the indirect calorimeter used in embodiments of the present invention comprises a respiratory connector such as a mask or mouthpiece, so as to pass respiration gases as the subject breathes; a flow pathway between the respiratory connector and a source and sink of respiratory gases (such as the atmosphere) which receives and passes the respiration gases; a flow meter configured to generate electrical signals as a function of the instantaneous flow of respiration gases passing through the flow pathway, such as an ultrasonic flow meter; and a component gas concentration sensor, such as a fluorescent oxygen sensor, which generates electrical signals as a function of the instantaneous fraction of gases such as oxygen and/or carbon dioxide in the respiration gases they pass through the flow pathway, such as the indirect calorimeter described above. Other oxygen sensor technologies can be used, for example based on thermal, chemical, optical, surface, electrical, or magnetic effects. The user's resting metabolism can be measured at repeated time intervals using the indirect calorimeter. The user breathes a multiple of inhalations and exhalations through the indirect calorimeter, so that the inhaled air and exhaled gas passes through the indirect calorimeter, the inhaled air volume and the exhaled flow volume are integrated with the instantaneous concentration of oxygen, and so the exhaled, inhaled, and consumed oxygen are determined. The component gas concentration sensor can be omitted if the molecular mass of respired gases is determined using an ultrasound method, in which case oxygen volumes consumed can be determined using ultrasound without a component gas sensor. Other indirect calorimeters can be used in embodiments of the present invention, for example such as described in U.S. Pat. Nos. 4,917,104; 5,038,792; 5,178,155; 5,179,958; 5,836,300, and 6,135,107 all to Mault, which are incorporated herein in-their entirety by reference. The indirect calorimeter can also be a module which interfaces with the PDA. The display, buttons, and process capabilities of the PDA are used to operate the module, display instructions for use of the indirect calorimeter, initiate tests, and record data.

For different exercise conditions, points per unit time can be scaled according to appropriate equations. For example, the additional energy expenditure due to treadmill use is often stated to be proportional to treadmill speed. Hence, if a certain point value is achieved in fifteen minutes at two mph, it can be assumed that twice that point value is achieved for twice the treadmill speed.

The points per unit time, or per exercise repetition, or per other unit of exercise, can be established for a variety of exercises, such as cycling, running, running on the spot, jogging, walking, swimming, skiing, and the like. The activity point expenditure can be adjusted according to speed, number of repetitions, exercise intensity, distance, or other appropriate activity level parameter.

Individual Energy Expenditure Equations

A person is asked to run on a treadmill while wearing a mask connected to an indirect calorimeter. The treadmill activity can then be converted to total energy expenditure using the indirect calorimeter data. The energy expenditure can be measured as a function of treadmill speed and treadmill gradient.

There are equations for VO₂ (or equivalently metabolic energy burning) known to those skilled in the exercise science arts. For example, for walking on a treadmill, there are equations of the form:

TEE=A+Bsg (equation 1)

where A is a constant related to resting metabolism (which is also accurately measured using an indirect calorimeter), s is the speed of the treadmill, g is the grade (or gradient) of the treadmill, and B is a constant. Values of B may be found in exercise and sports medicine books, but these values are generalized for an average person. By measuring treadmill energy expenditure as a function of speed and/or grade using the indirect calorimeter, a more accurate equation for the individual person can be obtained. This equation need not be linear in s and g. The equation could then, for instance, be stored in the person's portable computing device and used to calculate energy expenditure for future treadmill activity.

More generally, certain exercises and activity have one or more variable activity levels. For example, in treadmill use, the speed of the treadmill and its gradient are variable activity levels. In cycling, speed, wind resistance, and gradient are variable activity levels. In swimming, speed, stroke type, water salinity, and water temperature are variable activity levels. In the case of running or walking, the speed is the principal variable activity level. Hence, a pedometer may be calibrated to metabolic rate.

Using the indirect calorimeter, the energy expended in any activity having a variable activity level can be quantified. The person performs the activity for at least one value of the activity level (e.g. speed for a walking, running, or cycling exercise), the metabolic rate of the person is determined using an indirect calorimeter for each value of the activity level (e.g. speed) that the exercise is performed at; and then an equation is determined to relate the measured metabolic rate to the activity level (e.g. speed) at which the activity has been performed. The equation can then be used in the future to determine metabolic rate for the person as a function of the activity level. For example, if cycling in calm, flat conditions, metabolic rate can be determined as a function of cycling speed. An equation can then be devised to relate metabolic rate, and hence rate of activity point expenditure, to cycling speed. In the future, in similar conditions, the indirect calorimeter is not needed to find a good estimate of the metabolic rate or activity point expenditure as a function of cycling speed, as a determined calibration equation can be used.

Hence, a method of calibrating an activity monitor for a person comprises: attaching an activity monitor to the person; having the person engage in an activity; obtaining an activity signal from the activity monitor correlated with the activity level of the person; determining a metabolic rate for the person during the activity using an indirect calorimeter; and determining a correlation between the activity signal and the metabolic rate of the person performing the activity. The metabolic rate of a person and activity point expenditure for an exercise can then determined from the activity signal, using the correlation determined between the activity signal and the measured metabolic rate. A method of calculating energy expended during an activity having a variable activity level comprises the steps of: having the person perform the activity for at least one value of the activity level; determining a metabolic rate for the person for each value of the activity level; and determining an equation relating the metabolic rate to the activity level. The equation can be used to determine the metabolic rate and activity point expenditure for the person during the activity, knowing the activity level at which the person is performing the activity.

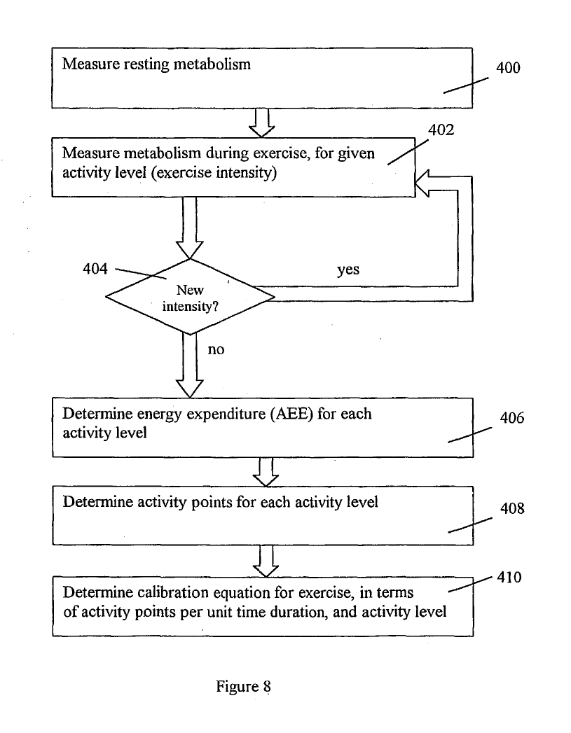

FIG. 8 is a flowchart showing a method by which an equation in activity points can be established for a given activity. Box 400 corresponds to measurement of resting energy expenditure. Box 402 corresponds to the measurement of total energy expenditure during the exercise. Box 404 corresponds to the possibility of repeating the exercise under different conditions such as at a higher activity level. Box 406 corresponds to the determination of AEE for each exercise activity level. Box 408 corresponds to the determination of points for each exercise activity level. Box 410 corresponds to the determination of a calibration equation for the exercise, in terms of activity points and exercise activity levels.

Numerical fitting of the activity point expenditure per unit time against activity level can be performed. For example, quadratic, cubic, or higher-order quadratic equations can be fitted to point expenditure versus speed data for e.g. a running activity. The fitted equation is quality checked for possible unacceptable behavior, which may include predicted infinite energy expenditures, discontinuities, and falling energy expenditures for higher activity levels.

For example, the rate of point achievement can be determined for different speeds of treadmill use. If the relationship is found to be linear, a linear equation can be used for future use of the treadmill, by which the treadmill speed is translated into the rate of point achievement. However, on an individual basis, it may be found that the relationship is nonlinear. In this case a quadratic equation, or some other numerical fit to the data, can be used to provide the most accurate translation of treadmill use to activity points. For example, an equation of the form TEE=A+Bsg+Cs²g+Dsg²+Es²g² can be derived with coefficients A, B, C, D, and E determined for a specific individual, using metabolic measurements at made at rest and during exercise using an indirect calorimeter. A related equation can then be readily defined in terms of activity points.

Feedback System Using an Interactive Television

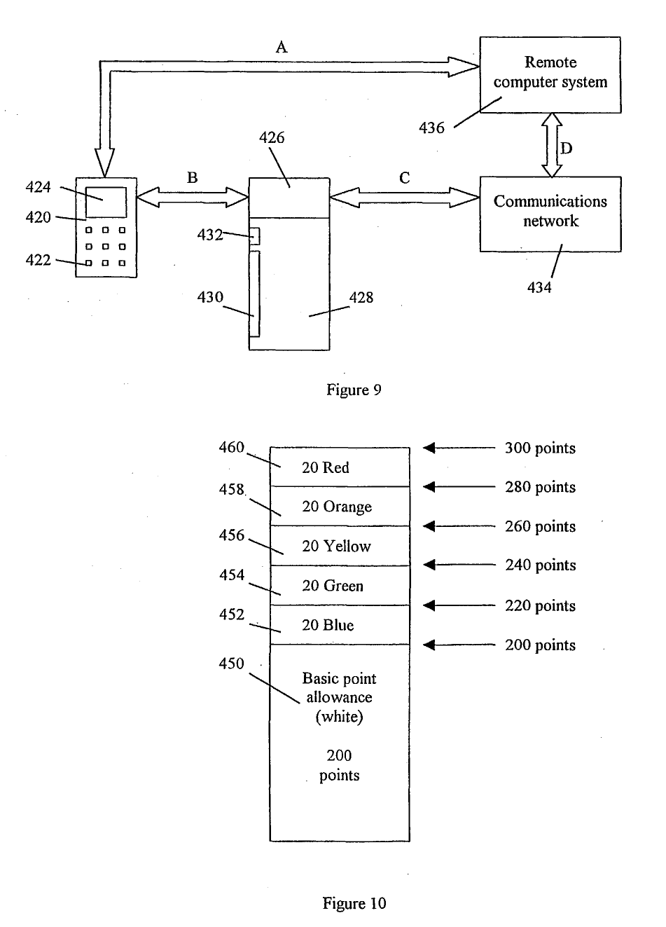

FIG. 9 shows a feedback system using an interactive TV which can be used with the present system. The portable electronic device 420 has a data entry mechanism 422 and a display 424. The device 420 can be a portable computing device, other portable electronic device, and can also be a modified remote control unit for the interactive TV. The function of remote control unit can be combined with other functions, such as computer, wireless phone, calculator, and the like. For example, an electronic device can be adapted to calculate the calorie balance for the person in terms of diet points, and this device can also be used as a remote control.

FIG. 9 also shows an interactive television 428 having a display 430 and speaker 432 connected to a set top box 426 which is connected over a communications link C to a communications network 434 and hence through a communications link D to a remote computer 436. The portable device 422 is shown having a communications link B to the set top box 426, and a communications link A to the remote computer.

The diet and exercise related data can be transmitted to the remote computer system 436 over a relatively slow link. Hence, the communications link A shown in FIG. 9 can be a cable phone line, a wireless phone link, DSL line, ISDN line, or other link. The feedback provided to the person via the interactive TV 428 can be content rich, compared with the diet and exercise data provided by the person, and this data can be is received over a relatively broad-band communications networks and communication links C and D. Communication links C and D can comprise fiber optics, cables, a wireless network, or a combination. Data uplinked from device 426 to 436 through link A can be transmitted at a lower data transmission rate than for the content rich information downloaded (received) by set top box 426 from device 436 (through links D and C). The communications link B is preferably a wireless links (such as IR or Bluetooth protocol), and can be used to transmit data to the remote computer system through the set-top box, act as a remote control, and to select menu options displayed on the screen 430, for example for ordering products.

The person transmits diet points related to consumption, nutritional data, activity points, resting points, and other data as appropriate to the remote computer system 436. Additional data can also be transmitted, such as weight, medical status, medicines consumed, and other information. The feedback to the person displayed on the interactive TV can comprise dietary advice, exercise suggestions, exercise programs, and the like. The feedback can also include suggested meal plans, which can include suggested deliveries of prepackaged food and meals, for example as supplied by a weight control business. The person can authorize delivery and payment for these prepackaged meals using the remote control.

Activity Zones

Activity points can be used to assist a person achieve training, cardiac rehabilitation, exercise, health maintenance, and other health or weight related goals. Activity zones can be defined relative to resting energy expenditure. FIG. 10 shows a possible graphical representation of a person's energy expenditure, and corresponding activity zones. The color labels correspond to a possible color graphic display on the display of an electronic device, or colored lamps illuminated on a feedback device. The basic region 450 (gray) corresponds to a person's resting energy expenditure. (In other embodiments, the gray zone can correspond to the sum of REE and energy expended during essential activities, which may be termed SEE or sedentary energy expenditure). The blue zone 452 corresponds to an activity zone, or range of activity energy expenditure over which the person expends zero to twenty activity points. In this example this corresponds to an AEE of 0-10% of REE, or a TEE of 200 to 220 activity-points. Other zones shown include green 454, yellow 456, orange 458, and red 460, corresponding to activity zones (or ranges) of different activity point expenditure. The activity zone boundaries are separated by ten percent of the resting energy expenditure. Other predetermined percentage ranges, or absolute value ranges, can be used according to personal goals.

During an exercise, a person can carry an activity sensor displaying a symbol, color, sounding a noise, or vibrating according to the activity zone that the person has achieved. The person can also be alerted to the transition between zones by visual or audio signals. A bar graph, for example as a graphic on an electronic display, or formed by a plurality of lamps, can provide a visual representation of activity energy expenditure to the person.

For example, the person can be trying to achieve a target zone, and visual, audio, tactile, or other feedback can be used to show the progress that the person is making. For example, a bar chart display can be shown on the display of an electronic device, for example showing different colors indicating progress towards the target zone.

Target activity zones can also be visually represented using letters, numbers, other characters, names, other colors, oscillation frequencies, and the like.

Other Methods of Calculating Activity Points

Activity points can also be calculated as a fraction of the person's resting energy expenditure REE. For example, an activity point can be defined as being 1% of REE. The person can then be encouraged to achieve a certain number of activity points per day. The zone system described above can be used for further motivation. For example, zones A to F can designate zero to five, five to ten, ten to fifteen, fifteen to twenty, and twenty to twenty-five points respectively. Other characters and symbols can be used.

This method is very useful for encouraging a person to achieve certain levels of exercise per day. An advantage of scaling the activity points to REE is that the person is encouraged to achieve higher levels of energy expenditure as their REE increases, for example due to buildup of muscle tissue.

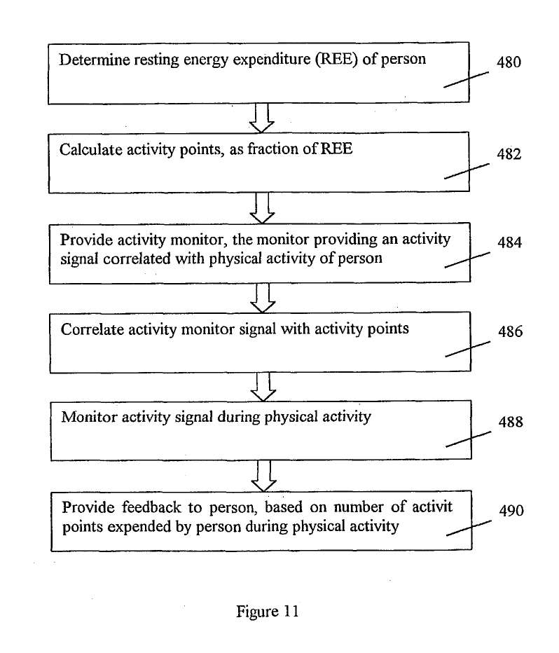

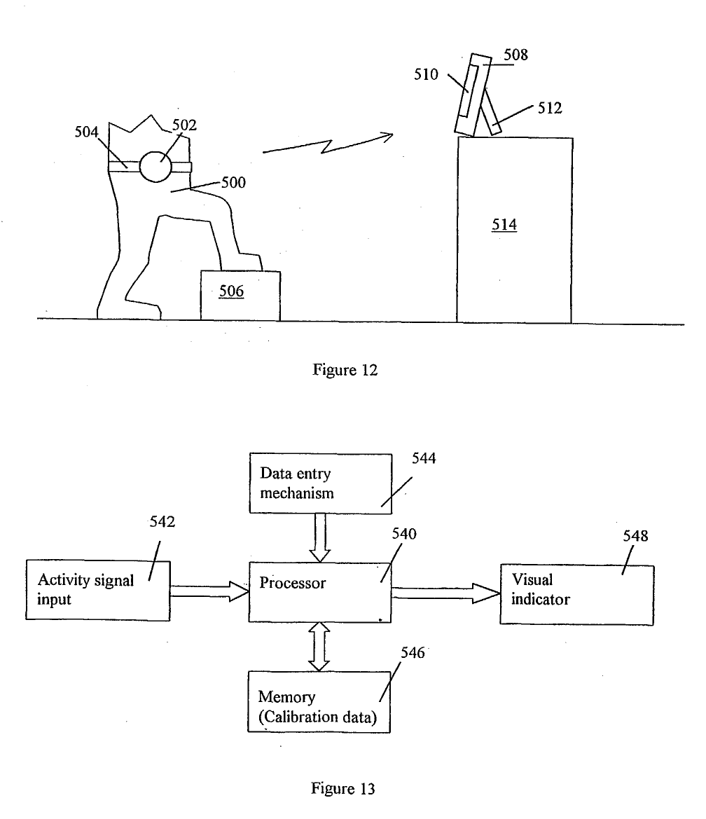

FIG. 11 illustrates a flow chart corresponding to a method for using activity points to provide feedback and encouragement to a person during an exercise program. Box 480 corresponds to measurement of REE for example using an indirect calorimeter. Box 482 corresponds to the calculation of the magnitude of activity points based on REE. Box 484 corresponds to providing an activity monitor to the person, which provides a signal correlated with the physical activity of the person. Box 486 corresponds to the correlation of the activity monitor signal with activity points. Box 488 corresponds to the monitoring of the person during an activity. Box 490 corresponds to the provision of feedback to the person based on the activity points achieved. FIG. 12 illustrates a person 500 performing a step exercise while, wearing an activity monitor 502 mounted on a belt 504. The step is shown at 506. The activity monitor transmits a signal to a portable computing device 508 shown located so as to provide a display 510. The device can be provided with a stand 512, to stand on shelf 514. The device can also in communication with an exercise, so as to receive exercise activity levels, repetitions, or other data

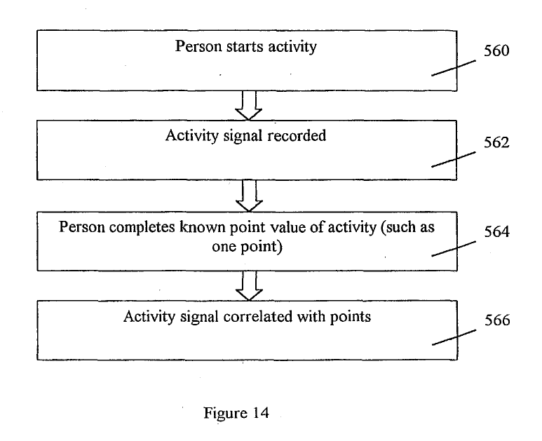

FIG. 13 shows a schematic of a unitary device which can be used to provide feedback to a person. The device comprises a processor 540, an activity sensor 542, a data entry mechanism 544, a memory containing calibration data 546, and a visual indicator such as a display or bar graph 548. This device can be used to provide a visual indication of activity preformed in terms of activity zones or points expended. Such a device can be calibrated using a method illustrated in FIG. 14. FIG. 14 is a flowchart corresponding to a calibration method. Box 560 corresponds to starting an activity. Box 562 corresponds to the monitoring of an activity signal. Box 564 corresponds to the person completing an activity point of activity, or some known multiple or fraction of an activity point. Box 566 corresponds to correlating the activity signal detected with the point value of the activity just performed. This can be achieved using the data entry mechanism shown in FIG. 13 (544).

General Activity Monitoring System

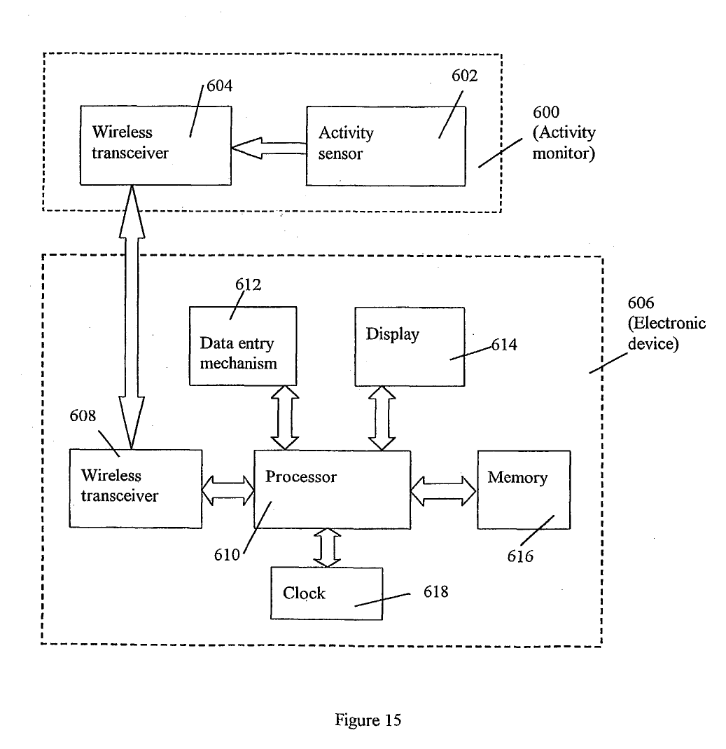

FIG. 15 shows a schematic of an activity monitoring system that can be used in embodiments according to the present invention. FIG. 16 shows an activity monitor 600, comprising an activity sensor 602 and a wireless transceiver 604. The system also comprises a portable electronic device 606 comprising a processor 610, a data entry mechanism 612, a display 614, a memory 616, and a clock 618.

Preferably the activity monitor 600 is worn on a belt, and the electronic device 606 is a portable computing device, such as a PDA, carried by the person. During exercise, the PDA can be placed nearby, in wireless range, to receive activity signals generated by the activity monitor. The PDA can receive transmissions, or manually entered data, barcode scans from other equipment such as exercise machines. Software on the PDA receives the data and converts the received data to diet and/or activity points.

Other Embodiments.

In some cases, it is desirable for a person to exercise near the anaerobic threshold. The Gas Exchange Monitor can be used to detect the anaerobic threshold, and this level can be included into the correlation of an activity monitor signal using the GEM. A person may receive an enhanced level of activity points for exercise at a desired activity level, such as near the anaerobic threshold. Fat burning can also be detected, using respiratory quotient data provided by the GEM, or using a ketone sensor providing a signal correlated with ketone and aldehyde levels in exhaled breath. A person can receive an increased number of activity points for activities which induce increased fat metabolism. An indirect calorimeter can be combined with a heart rate monitor so that heart rate can be correlated with energy expenditure or activity points expenditure.

Other embodiments will be clear to those skilled in the arts. The invention is not to be limited by the examples given above. Having described my invention, I claim:

1. A method of assisting a person to achieve a weight control goal, the method comprising:
determining a resting energy expenditure for the person using an indirect calorimeter;
assigning resting points to the resting energy expenditure of the person;
assigning activity points to an activity level of the person;
monitoring consumption of consumables by the person, and assigning consumption points to the consumables consumed, wherein the consumption points are assigned based on the calorie content and at least one other nutritional parameter of the consumables;
determining the a difference between the consumption points and a summation of the expenditure points with the activity points; and
providing feedback to the person based on the difference.

2. The method of claim 1, wherein the consumption points are assigned based on the calorie content and fat content of the consumables.

3. The method of claim 1, wherein the resting points are assigned based on the calorie value of the resting energy expenditure and a fat content of the person's diet.

4. The method of claim 1, wherein the activity points are assigned based on the calorie value of the activity and a fat content of the person's diet.

5. The method of claim 1, wherein the activity level is determined using a portable activity monitor.

6. A system for assisting a person in a weight control program, the system comprising:
an activity monitor having an activity sensor and a transmitter, wherein the activity monitor transmits an activity signal correlated with a physical activity level of the person;
a portable electronic device having a processor, a display, a receiver, and a memory, wherein the portable electronic device receives the activity signal from the activity monitor; and
a software application program running on the portable electronic device, adapted to receive data corresponding to consumables consumed by the person, to assign diet points to the consumables consumed based on calorie content and at least one other nutritional parameter, to assign activity points correlated with the activity signal, to receive a resting energy expenditure of the person, to assign resting points correlated with the resting energy expenditure, and to provide a visual representation of the difference between the diet points and the sum of the resting points and the activity points on the display.

7. A method of assisting a person to achieve a level of physical activity, the method comprising:
determining a resting energy expenditure of the person;
assigning a number of activity points to the calorie value of the resting energy expenditure, so that a single activity point is a fraction of the person's resting energy expenditure;
monitoring an activity level of the person;
correlating the activity level of the person with a value of activity points expended; and
providing feedback to the person correlated with the value of activity points expended.

8. The method of claim 7, wherein the resting energy expenditure of the person is determined using an indirect calorimeter.

9. The method of claim 7, wherein the feedback comprises a graphic on an electronic display.

10. The method of claim 7, wherein the feedback comprises the illumination of colored lights.

11. A system to assist a person achieve a level of energy expenditure, the system comprising:
an activity sensor;
a processor, receiving an activity signal from the activity sensor, adapted to determine an energy expenditure by the person from the activity signal, and to generate a visual representation on a visual indicator of a predetermined range of energy expenditure within which the energy expenditure falls.

12. The system of claim 1, wherein the predetermined range of energy expenditure is calculated based on the resting energy expenditure of the person.

13. The system of claim 11, wherein the visual indicator is an electronic display.

14. The system of claim 11, wherein the visual indicator comprises a plurality of colored lights.

15. The system of claim 11, wherein the activity sensor is a heart rate sensor.

16. The system of claim 11, wherein the activity sensor is a pedometer.

17. The system of claim 11, wherein the activity sensor is a position location device.

18. The system of claim 11, wherein the activity sensor is an indirect calorimeter.

19. The system of claim 11, wherein the activity sensor comprises part of an exercise machine.
